(19) United States
(12) Patent Application Publication
(10) Pub. No.: US 2014/0335019 A1
(43) Pub. Date: Nov. 13, 2014

(54) COMPOSITIONS AND METHODS FOR THE TREATMENT AND ANALYSIS OF NEUROLOGICAL DISORDERS

(71) Applicants: THE REGENTS OF THE UNIVERSITY OF MICHIGAN, Ann Arbor, MI (US); THE UNIVERSITY OF BRITISH COLUMBIA, Vancouver (CA)

(72) Inventors: Mi Hee Lim, Ulsan (KR); Katayoun Saatchi, Vancouver (CA); Urs Hafeli, Vancouver (CA); Joo-Yong Lee, Seoul (KR)

(21) Appl. No.: 14/362,339

(22) PCT Filed: Nov. 30, 2012
(86) PCT No.: PCT/US12/67413

§ 371 Date: Jun. 2, 2014

Related U.S. Application Data
(60) Provisional application No. 61/566,356, filed on Dec. 2, 2011.

(51) Int. Cl. C07D 215/06; A61K 51/04; G01N 33/50; C07D 213/53; C07D 213/38; C07D 213/42; C07D 471/04; C07D 215/48

(52) U.S. Cl. CPC C07D 215/06; C07D 471/04; A61K 51/0455; C07D 215/48; C07D 213/53; C07D 213/38; C07D 213/42; G01N 33/5014
USPC 424/1.89; 546/121; 514/300; 424/1.85; 546/171; 514/311; 546/170; 546/286; 514/344; 546/329; 514/357; 534/770; 514/150; 435/29

(57) ABSTRACT

Provided herein are compositions and methods for the treatment and analysis of neurological disorders. In particular, provided herein are small molecules targeted to amyloid-β (Aβ) or metal-Aβ species for the treatment, diagnosis, or study of neurological conditions such as Alzheimer's disease (AD) and other diseases and conditions.

### FIELD

Provided herein are compositions and methods for the treatment and analysis of neurological disorders. In particular, provided herein are small molecules targeted to amyloid-β (Aβ) or metal-Aβ species for the treatment, diagnosis, or study of neurological conditions such as Alzheimer's disease (AD) and other diseases and conditions.

### BACKGROUND

More than 36 million people worldwide have Alzheimer's disease (AD), a devastating and fatal form of neurodegeneration that is the sixth leading cause of death in the United States. This number is expected to exceed 80 million by 2040. AD patients experience multiple cognitive deficits including memory loss and disorientation, which are caused by a breakdown of neuronal function and progressive neuronal cell death. Unfortunately, despite the availability of drugs with modest symptomatic benefit, no therapeutic approach to date has been shown to slow down or prevent the disease. Although the pathology of AD is known, its etiology is still only partially understood, which has hampered therapeutic development. The key pathological markers in AD are amyloid-β (Aβ) plaques and neurofibrillary tangles, the accumulation of which is accompanied by oxidative stress, inflammation, and neurodegeneration. The widely accepted “amyloid hypothesis” in AD states that Aβ is a proximal causative agent. It remains controversial, however, which forms of Aβ, from small oligomers to large fibrils, are central to AD pathogenesis. In addition to Aβ aggregate deposits, dyshomeostasis and miscompartmentalization of metal ions occur in AD brains. Some studies show that metal ions play a role in Aβ aggregate deposition and neurotoxicity in the brain, leading to AD.

### SUMMARY

Provided herein are compositions and methods for the treatment and analysis of neurological disorders and other diseases and conditions. In particular, provided herein are small molecules targeted to amyloid-β (Aβ) or metal-Aβ species for the treatment, diagnosis, or study of neurological conditions such as Alzheimer's disease (AD) and other diseases and conditions. In various embodiments herein, the small molecules (a) target metal-Aβ species and modulate their interaction/reactivity in the brain (utilization as chemical tools or therapeutics) and/or (b) detect Aβ or metal-Aβ species in the AD brain (diagnostics/screening).

In some embodiments, the small molecules provided herein specifically target Aβ or metal-Aβ species. In some such embodiments, small molecules that target metal-Aβ species specifically and control metal-Aβ interaction have primary structural moieties for both metal chelation and Aβ interaction—they combine two functions, metal chelation and Aβ interaction in the same molecule. In some embodiments, the molecules contain tunable multifunctionality such as metal chelation, Aβ interaction, and antioxidant capability. In some embodiments, the geometry of small molecules is selected to control redox cycles of the metal center or for additional structural moieties to have antioxidant capabilities in order to attenuate ROS production from redox-active metal-Aβ species. The compounds find use as chemical tools to investigate metal-Aβ-involved events and help define the relationship between metal-Aβ interaction and AD neuropathogenesis, as well as diagnostic and therapeutic agents in AD and other neurological diseases and conditions.

In some embodiments, compounds are selected based on a rational structure-based design and tested for desired activities and properties in the appropriate assays and models. For example, in some embodiments, direct insertion of metal binding donor atoms into the structures of Aβ interacting molecules is employed (see e.g., FIG. 1, top). Furthermore, because of the potential brain applications, structures are selected to allow penetration of the blood-brain barrier (BBB). For example, the compounds may be selected with the restrictive terms of Lipinski's rules and calculated logBB (Low molecular weight (MW≦450); relatively lipophilic (clog P, calculated logarithm of the octanol/water partition coefficient, ≦5); hydrogen-bond donor atoms (HBD≦5); hydrogen-bond acceptor atoms (HBA≦10); small polar surface area (PSA≦90 Å²); and logBB=−0.0148×PSA+0.152×clogP+0.130 (logBB>0.3, readily cross the BBB; logBB<−1.0, only poorly distributed to the brain)).

Exemplary compounds provided herein (e.g., FIG. 1, bottom, 1a/b-series, 2a/b, 3a/b, 4a/b, 5a-5d; FIG. 9, compound 5 series) include structural features for metal chelation and Aβ interaction and reduced metal-induced Aβ aggregation and neurotoxicity including ROS generation in vitro and in living cells. Using this incorporation strategy, compounds are provided as chemical reagents to target metal-Aβ species and modulate metal-Aβ interaction/reactivity in vitro and in vivo while maintaining low molecular weight, which provides for their use for brain applications. It should be understood that various derivatives and analogs of the compounds described herein (e.g., FIG. 1, FIG. 9) are within the scope of the invention. For example, hydrogens on any of the ring positions may be substituted for labels (groups containing radionuclides or contrast agents, halogens, etc.), alkyl groups (e.g., methyl, ethyl, etc.), hydroxyl groups, amine groups, any of which may contain a radioactive atom, other functionalities, or side arms, etc., so long as the desired properties of the molecule are retained (e.g., Aβ interaction, metal chelation, BBB penetration)—such properties are deducible using assays described herein or otherwise known in the art. Likewise, atoms in the rings (carbons, nitrogens) may be substituted for different atoms or their positions within the rings altered. Linking groups between rings may be altered to add, remove, or move carbons, which may be substituated or unsubstituted.

In some embodiments, provided herein is a compound having the structure:

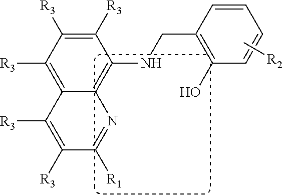

, wherein R₁ is selected from the group consisting of —H, —OH, —CH₃, —CO₂CH₃, —CO₂H, —CH₂OH, —F, and —O-glucose, R₂ is selected from the group consisting of —H, —OH, —NH₂, —NH(CH₃), —N(CH₃)₂, —F, and —O-glucose, and R₃ is each independently selected from the group consisting of —H, —OH, —NH₂, —NH(CH₃), —N(CH₃)₂, —(CH₂)ₙCH₃ (n=1-10), —F, and —O-glucose. Any of the R positions may comprise a radionuclide or further comprise a group (e.g., sugar, polyethylene glycol, alkyl, etc.) bearing a radioisotope. Variations of this structure are also contemplated, including those containing one or more nitrogens in the ring having the R₂ group (e.g., m or p position relative to the position with the hydroxyl group).

In some embodiments, such compounds have the structure:

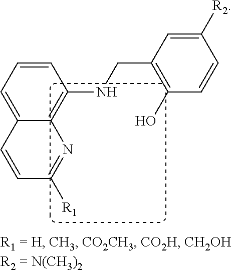

Further provided are compositions comprising such compounds (e.g., pharmaceutical compositions, e.g., further comprising a buffer, carrier, adjuvant, co-administered second therapeutic agent, etc.).

Further provided herein is a pharmaceutical composition comprising one or more of the following compounds

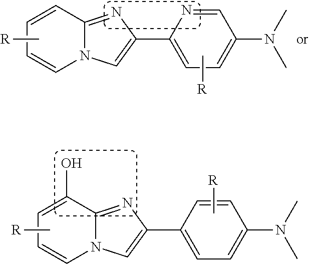

wherein R is selected from the group consisting of —H, —I, —F, or CH₃;

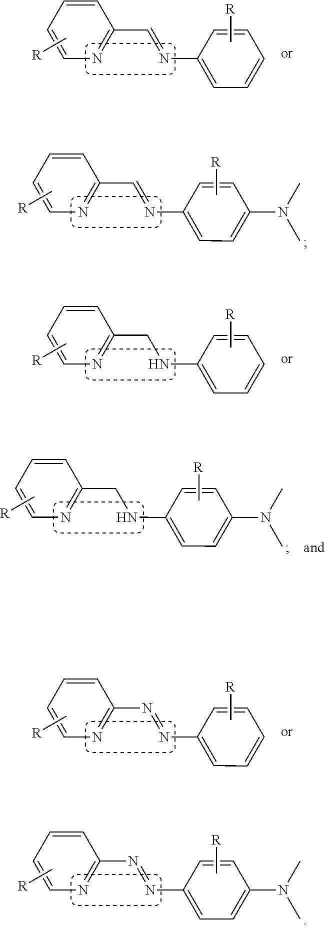

In some embodiments, provided herein are methods for the treatment of a subject, comprising administering any of the above compounds or compositions to a subject (e.g., a human subject, a mammal, a rodent, etc.). In some embodiments, the subject has or is at risk of acquiring (e.g., based on genetics, age, family history, etc.) a neurological disease or condition. In some embodiments, the neurological disease or condition is characterized by, associated with, or has a pathological marker of amyloid-β (Aβ) plaques. In some embodiments, the disease or condition is Alzheimer's disease.

In some embodiments, provided herein is the use of any of the compounds or compositions above (e.g., use for the treatment of any neurological disease or condition as described above). In some embodiments, provided herein is a method of manufacture of a medicament of any of the compositions described above for the treatment of any neurological disease or condition described above.

Also provided herein are pharmaceutical compositions comprising a compound having a metal chelation component and an amyloid β interaction component. In some embodiments, the compound penetrates the blood-brain barrier of a human.

Also provided herein are labeled compounds that find use in diagnostic methods and uses. In some such embodiments, any of the compounds described above may further comprise a label (e.g., a radionuclide, a contrast agent (e.g., iodine, barium, gadolinium, etc.), an optical label, or any other atom or other moiety that can be directly or indirectly detected in vitro, in situ, or in vivo).

### DESCRIPTION OF FIGURES

FIG. 1 (top) shows a schematic of the incorporation approach of small molecule design employing an Aβ interaction component and a metal chelation component; and (bottom) exemplary small molecule compounds using such an approach.

FIG. 2 shows the results of 2D NMR and docking studies of Aβ with compound 3b: (a) Overlay of 2D TROSY ¹H-¹⁵N HSQC spectra of Aβ upon addition of 3b. The expanded region (red box) depicts significant chemical shifts (see Detailed Description, below). Methods: Spectra recorded on a 900 MHz Bruker Avance NMR spectrometer. Five (red), ten (blue), and 15 (green) equivalents of 3b were added to the ¹⁵N-labeled Aβ₁₋₄₀ (black, 200 mM SDS-d₂₅, 20 mM NaPi, pH 7.3, 25° C.). (b) Chemical shift perturbations (10 equiv. 3b). *Denotes absent or overlapped signals. (c) Docking studies of Aβ (PDB 1BA4) and 3b using software AutoDock4.

FIG. 3 shows chemical structures of compounds useful in imaging experiments and assay. These compounds find use for imaging using, for example, PET or SPECT or CT or MR imaging.

FIG. 4 shows a schematic overview of inhibition and disaggregation experiments to analyze the formation and disaggregation of structures comprising Aβ and assembled from Aβ monomers.

FIG. 5 shows an overview and results of investigations of small molecules toward metal-Aβ species. FIG. 5A: TEM images of CuII-treated Aβ incubated with compounds 1a/b, 3a/b, and clioquinol (CQ). FIG. 5B: Interaction of 3b with human AD brain tissue homogenates. Visualization of proteins including Aβ species by silver staining (lane 1) or native gel electrophoresis using Western blotting (6E10) (supernatant untreated (lane 2) and incubated with 3b for 24 h (lane 3)).

FIG. 6 shows the results of cytotoxicity studies of metal (CuII or ZnII)-associated Aβ species with the compounds in M17 cells using the MTT assay. These results indicate that 3a/b are capable of reducing metal-Aβ neurotoxicity in living cells. Treatment of Aβ in the absence and presence of metal ions for 24 h results in ca. 90, ca. 70 (for CuII), and ca. 80% (for ZnII) survival of cells, respectively. Methods: M17 cells were seeded in 96 well plate followed by introduction of Aβ (20 μM), metal ions (20 μM), compound (40 μM) 24 h later. Then, the cells were incubated for 24 h.

FIG. 7 shows the results of biodistribution experiments of radiolabeled compound 3b.

FIG. 8 shows synthesis schemes for exemplary compounds.

FIG. 9 shows exemplary compounds.

### DEFINITIONS

To facilitate an understanding of the technology described herein, a number of terms and phrases are defined below.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. In certain other embodiments, a straight chain or branched chain alkyl has 1 to 6 carbon atoms in its backbone. Likewise, cycloalkyls have from about 3 to about 10 carbon atoms in their ring structure, and alternatively about 5, 6 or 7 carbons in the ring structure. Unless specified otherwise, alkyl groups are optionally substituted with halogen, alkoxy, hydroxyl, or amino In certain embodiments, the alkyl group is not substituted, i.e., it is unsubstituted. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “haloalkyl” refers to an alkyl group that is substituted with at least one halogen. For example, —CH₂F, —CHF₂, —CF₃, —CH₂CF₃, —CF₂CF₃, and the like.

The term “alkylene” as used herein refers a straight or branched, saturated aliphatic, divalent radical. Exemplary alkylene groups include methylene (—CH₂—), ethylene (—CH₂CH₂—), trimethylene (—CH₂CH₂CH₂—), and the like.

The term “aralkyl” refers to an alkyl group substituted with an aryl group.

The term “heteroaralkyl” refers to an alkyl group substituted with a heteroaryl group.

The terms “alkenyl” and “alkynyl” are art-recognized and refer to unsaturated aliphatic groups analogous in length and possible substitution to the alkyls described above, but that contain at least one double or triple bond respectively. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl, etc. The term “cycloalkenyl” is art-recognized and refers to cyclic aliphatic group containing at least 1 C—C double bond. Unless specified otherwise, cycloalkenyl groups are optionally substituted with halogen, alkyl, alkoxy, hydroxyl, or amino In certain embodiments, the cycloalkenyl group is not substituted, i.e., it is unsubstituted. Exemplary cycloalkenyl groups include cyclohexenyl and cyclopentenyl.

The term “aryl” is art-recognized and refers to a carbocyclic aromatic group. Representative aryl groups include phenyl, naphthyl, anthracenyl, and the like. Unless specified otherwise, the aromatic ring is substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “aryl” also includes polycyclic ring systems having two or more carbocyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, and/or aryls. The term “haloaryl” refers to an aryl group that is substituted with at least one halogen. In certain embodiments, the aromatic ring is substituted with halogen, alkoxy, hydroxyl, or amino In certain embodiments, the aryl group is not substituted, i.e., it is unsubstituted.

The term “monocarbocyclic aryl” is art-recognized and refers to a carbocyclic, single-ring aromatic group, i.e., phenyl. Unless specified otherwise, the monocarbocyclic aryl is optionally substituted with one or two occurrences of halogen, methyl, ethyl, propyl, phenyl, pyridinyl, hydroxyl, amino, or acyl. In certain embodiments, the monocarbocyclic aryl group is not substituted, i.e., it is unsubstituted.

The term “heteroaryl” is art-recognized and refers to aromatic groups that include at least one ring heteroatom. In certain instances, a heteroaryl group contains 1, 2, 3, or 4 ring heteroatoms. Representative examples of heteroaryl groups includes pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring is substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaromatic ring is substituted with halogen, alkoxy, hydroxyl, or amino. In certain embodiments, the heteroaryl group is not substituted, i.e., it is unsubstituted.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

As used herein, the term “heterocyclic” represents, for example, an aromatic or nonaromatic ring containing one or more heteroatoms. The heteroatoms can be the same or different from each other. Examples of heteratoms include, but are not limited to nitrogen, oxygen and sulfur. Aromatic and nonaromatic heterocyclic rings are well-known in the art. Some nonlimiting examples of aromatic heterocyclic rings include pyridine, pyrimidine, indole, purine, quinoline and isoquinoline. Nonlimiting examples of nonaromatic heterocyclic compounds include piperidine, piperazine, morpholine, pyrrolidine and pyrazolidine. Examples of oxygen containing heterocyclic rings include, but not limited to furan, oxirane, 2H-pyran, 4H-pyran, 2H-chromene, and benzofuran. Examples of sulfur-containing heterocyclic rings include, but are not limited to, thiophene, benzothiophene, and parathiazine. Examples of nitrogen containing rings include, but not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. Unless specified otherwise, the heterocyclic ring is optionally substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like.

The term “heterocycloalkyl” is art-recognized and refers to a saturated cyclic aliphatic group containing at least one N, O, or S ring atom. The term “heterocycloalkyl” also includes bicyclic ring systems in which two or more atoms are common to two adjoining rings, where both rings are saturated and at least one of the rings contains a N, O, or S ring atom. Unless specified otherwise, heterocycloalkyl groups are substituted with 1, 2, or 3, substituents independently selected from the group consisting of alkyl, halogen, alkoxy, hydroxyl, amino, and —C(O)alkyl. In certain embodiments, the heterocycloalkyl group is substituted with 1 substituent selected from the group consisting of alkyl, halogen, alkoxy, hydroxyl, amino, and —C(O)alkyl. In certain embodiments, the heterocycloalkyl group is not substituted, i.e., it is unsubstituted.

Certain compounds contained in compositions, or their precursors, described herein may exist in particular geometric or stereoisomeric forms. Unless stated otherwise, all such compounds, including cis- and trans-isomers, R- and S-enantiomers, diastereomers, (D)-isomers, (L)-isomers, the racemic mixtures thereof, and other mixtures thereof, are contemplated as included herein. Additional asymmetric carbon atoms may be present in a substituent such as an alkyl group.

The terms “individual,” “patient,” or “subject” are used interchangeably and include any animal, including mammals, preferably mice, rats, other rodents, rabbits, dogs, cats, swine, cattle, sheep, horses, or primates, and most preferably humans. The compounds described herein can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

As used herein, the term “effective amount” refers to the amount of a compound sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable salt” refers to any pharmaceutically acceptable salt (e.g., acid or base) of a compound which, upon administration to a subject, is capable of providing a compound or an active metabolite or residue thereof. As is known to those of skill in the art, “salts” of the compounds may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁻, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds compounded with a suitable cation such as Na⁺, NH₄⁺, and NW₄⁻ (wherein W is a C₁₋₄ alkyl group), and the like.

For therapeutic use, salts of the compounds are contemplated as being pharmaceutically acceptable. However, salts of acids and bases that are non-pharmaceutically acceptable may also find use, for example, in the preparation or purification of a pharmaceutically acceptable compound.

### DETAILED DESCRIPTION

Provided herein are compositions and methods for the treatment and analysis of neurological disorders and other diseases and disorders. In particular, provided herein are small molecules targeted to amyloid-β (Aβ) or metal-Aβ species for the treatment, diagnosis, or study of neurological conditions such as Alzheimer's disease (AD) and other diseases of conditions that involve or are associated with amyloid-β (Aβ) (e.g., diabetes). In various embodiments herein, the small molecules (a) target metal-Aβ species and modulate their interaction/reactivity (utilization as chemical tools or therapeutics) and/or (b) detect Aβ or metal-Aβ species (diagnostics/screening).

Various assay described below can be carried out as described in Choi et al., Proc. Natl. Acad. Sci., 107(51), 21990-21995 (and supplement) (2010), herein incorporated by reference in its entirety.

Preparation and Characterization of Small Molecules with or without Aβ

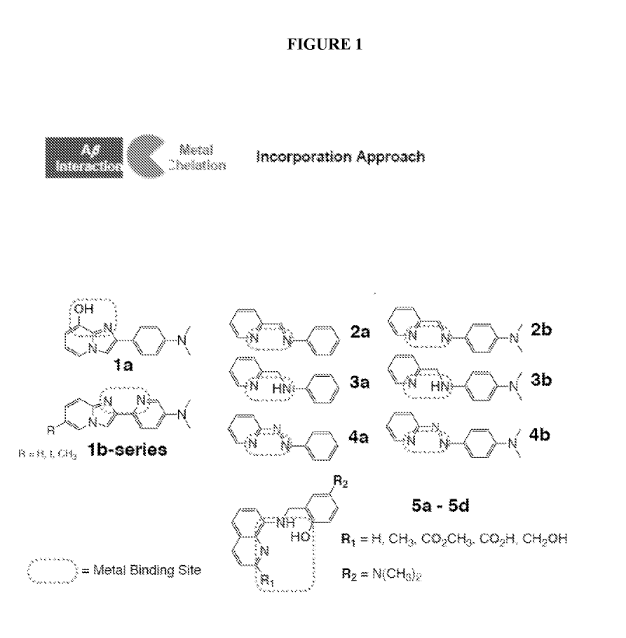

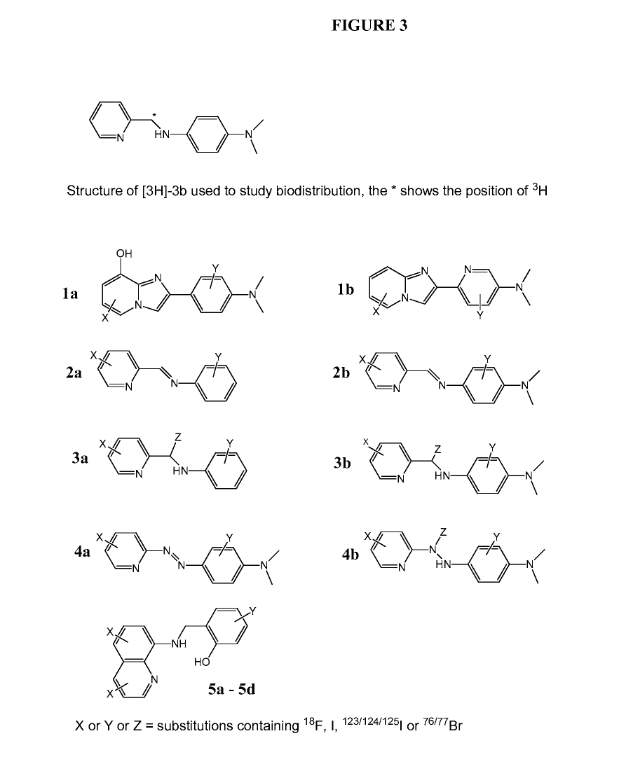

Utilizing the incorporation strategy, a series of initial exemplary compounds (FIG. 1) were designed and synthesized. The frameworks for these compounds were based on Aβ imaging probes (FIG. 3, IMPY=4-(6-iodoimidazo[1,2-α]pyridin-2-yl)-N,N-dimethylaniline (IMPY) and p-1-stilbene=(E)-4-(4-iodostyryl)-N,N-dimethylaniline), which show strong binding affinity to Aβ species. These imaging agents are small, neutral, lipophilic, and thus able to penetrate the BBB. Furthermore, they are easily removed from normal brain tissue and accumulate in the blood at relatively low levels, which reduces their toxicity for in vivo applications. For metal chelation, N and/or O donor atoms were directly incorporated into these Aβ interacting structures.

The initial molecules were prepared via cyclocondensation or Schiff base condensation, and/or reduction. Defined by the restrictive terms of Lipinski's rules and calculated logBB, the compounds are contemplated to find use in the brain (for example: for 1a, MW=253.31, clogP=3.86, HBD=1, HBA=4, PSA=40.77, and logBB=0.12). As a measure of possible BBB permeability of the compounds, the parallel artificial membrane permeability assay (PAMPA-BBB) was performed, showing their potential BBB penetration via passive diffusion, compared to known BBB permeable compounds such as verapamil. Furthermore, from solution speciation and metal binding studies through UV-vis variable-pH titrations, at physiological pH (ca. 7.4), the neutral form of 3b existed mainly (neutral form is preferable for BBB penetration) and a mixture of 1:1 and 1:2 metal-ligand complexes were observed (binding stoichiometry). Also, from these titration studies, binding affinity of 3b for metal ions indicated high picomolar (for CuII) and low micromolar (for ZnII) at pHs 6.6 and 7.4, which is comparable to those reported for Cu-Aβ (picomolar to nanomolar) and Zn-Aβ (micromolar). Therefore, it is contemplated that this compound competes for metal ions from soluble Aβ species. Lastly, metal selectivity studies using 3b show that this compound is relatively selective for CuII over other divalent metal ions (e.g., CaII, MgII, MnII, FeII, CoII, NiII, and ZnII).

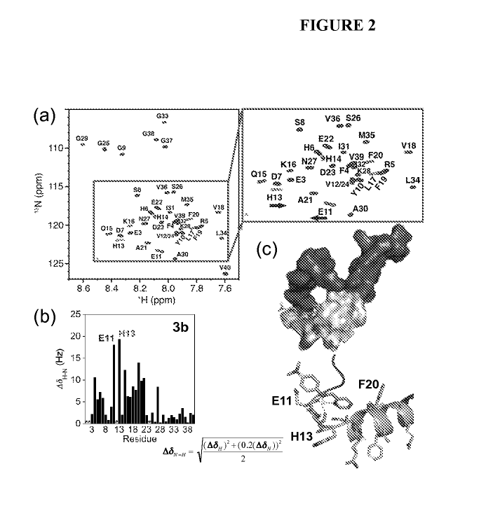

The direct interactions of the compounds with Aβ monomer without metal ions were investigated using high-resolution 2D TROSY ¹H-¹⁵N HSQC-based NMR structural determinations (900 MHz, TROSY=Transverse Relaxation Optimized Spectroscopy; HSQC=Heteronuclear Single Quantum Correlation) or IM-MS (mass spectrometry). Interestingly, upon treatment with 1a/b, 2a/b, or 3a/b chemical shifts of the Aβ residues E11 and H13 were significantly shifted. 1a/b, 2a/b, or 3a/b showed more influence on the less ordered, hydrophilic N-terminus portion of Aβ than the hydrophobic C-terminus, which is presented in the plot displaying the difference of ¹H-¹⁵N shifts (Δδ, Hz) as a function of the amino acid sequence (FIG. 2 for 3b). These observations reveal that 1a/b, 2a/b, or 3a/b interact with Aβ and have close contact with the metal coordination site of Aβ, where H6, H13, and H14 residues are involved for metal binding (the results by NMR were consistent with those by docking studies (AutoDock, FIG. 2)).

In addition, NMR studies of the compound (3b) and Aβ monomer pretreated with ZnCl₂ indicated that a ternary complex containing Aβ, ZnII, and the molecule may be generated, which could be responsible for its reactivity toward metal-Aβ species. While an understanding of the mechanism is not necessary to practice the invention and the invention is not limited to any particular mechanism of action, these observations imply that the compounds may target metal ions associated with Aβ species. In addition, 3b interaction with Aβ aggregates was also investigated employing an ELISA (molecules in contact with Aβ aggregates can block the antibody binding, which is detected optically). This ELISA study demonstrated that 3b interacted with Aβ aggregates, similar to ThT, a well-known fluorescent probe for Aβ aggregates. Overall, our NMR and ELISA investigations demonstrated direct interactions of the compounds with Aβ and metal-Aβ, and thus, they are classified as bifunctional small molecules (metal chelation and Aβ interaction).

### Preparation of Radiolabeled Small Molecules

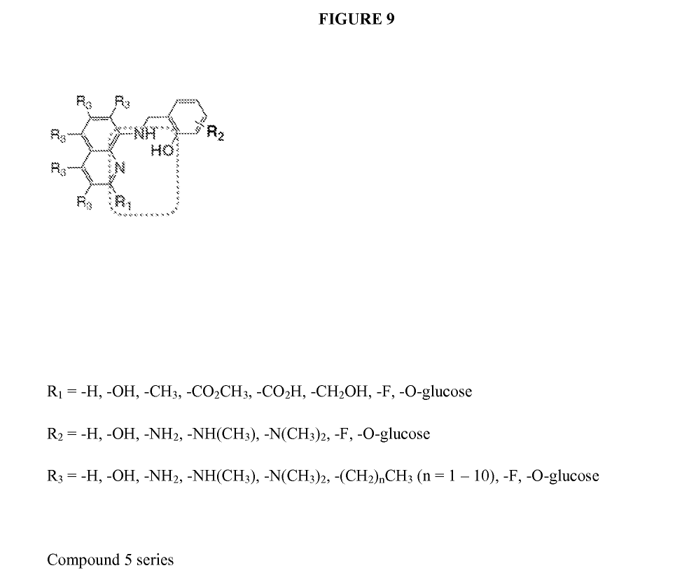

Radiolabeling is one of the routes to trace drugs in vivo. Depending on the radioisotope chosen, various modes of detection can then be used. For preliminary experiments to investigate crossing BBB, 3b was modified with ³H shown with *. Reductive ammination of 2b with tritiated sodium borohydride (NaB[³H]₄) results in 3b where one proton is substituted with a tritium (FIG. 3). This compound can be traced in vivo due to radioactivity but it cannot be imaged. To be able to use a PET, SPECT, CT or X-ray camera the small molecules can modified with ¹⁸F, different iodine isotopes, e.g., I ¹²³I, ¹²⁴I or ¹²⁵I, or different bromine isotopes such as ⁷⁶Br or ⁷⁷Br (FIG. 3). Similarly all structures in FIGS. 1 and 9 could be prepared with a label for imaging using various modalities like PET, SPECT, CT, X-ray and MR imaging.

Reactivity of Small Molecules with Aβ and Metal-Aβ Species

Metal-Induced Aβ Aggregation Influenced by Small Molecules

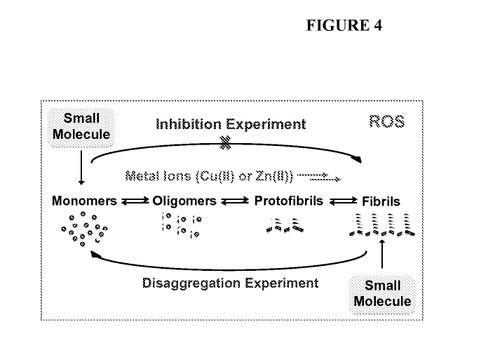

In vitro, metal binding to Aβ species has been studied and suggested to be linked to facilitation of Aβ aggregation. To investigate if/how the compounds (e.g., FIGS. 1 and 9) could affect metal (CuII or ZnII)-induced Aβ aggregation, two separate studies were performed (FIG. 4): inhibition (preventing the formation of metal-induced Aβ aggregates) and disaggregation (the transformation of metal-Aβ aggregates). The degree of Aβ aggregation was probed by transmission electron microscopy (TEM) and biological methods (e.g., native gel electrophoresis with Western blotting (6E10, anti-Aβ antibody)). Establishing the protocols for in vitro investigations, it was also found that general analytical methods (e.g., fluorescence and turbidity assays) can be employed to verify the degree of Aβ aggregation due to the interference of their analysis windows with the absorptions of compounds and their corresponding metal complexes.

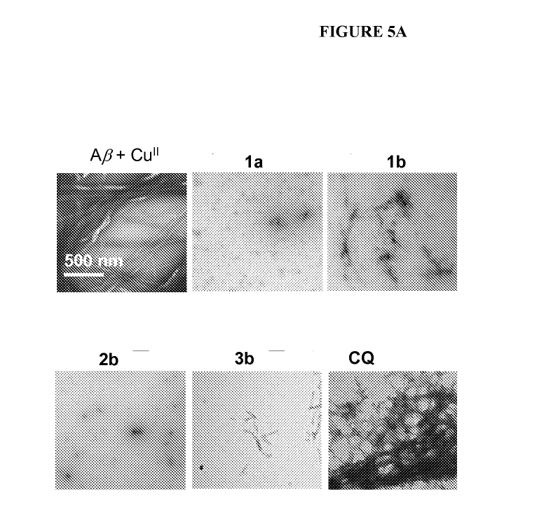

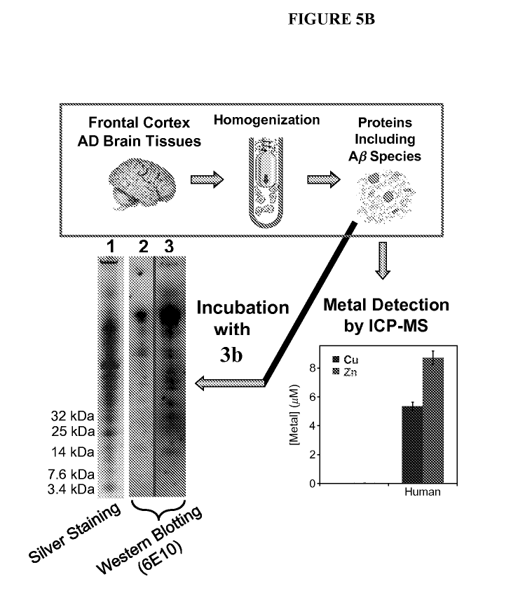

For the inhibition studies (FIG. 4), the metal-induced Aβ aggregation could be modulated by treatment with the compounds. As shown in TEM studies (FIG. 5, top, for CuII; data not shown for 1,10-phenanthroline (phen), ethylenediaminetetraacetic acid (EDTA), MPY, and the stilbene derivative), less metal-triggered Aβ aggregation was indicated in the presence of the compounds over the well-known metal chelators CQ, phen, and EDTA as well as the control molecules MPY and the stilbene derivative which do not contain a metal binding site. For the disaggregation studies (data not shown), a small molecule was allowed to react with Aβ aggregates, generated by incubating Aβ with CuII or ZnII for 24 h at 37° C. The compounds induced more disaggregation of Aβ aggregates, as compared to CQ, phen, and EDTA. Western blotting with a 6E10 antibody, confirmed the presence of a distribution of gel-permeable low molecular weight (LMW) species generated by both inhibition and disaggregation experiments using the compounds. As a control experiment, when metal-free Aβ species were incubated with compounds in both inhibition and disaggregation studies, Aβ fibrillogenesis was still observed, indicating that the molecules are specific for metal-Aβ species and if they are radiolabeled, they can be used as indicators for Aβ aggregates in the AD brain. Overall, interaction with either metal ions alone or Aβ alone by small molecules is not sufficient to modulate metal-induced Aβ aggregation. Synergistic interactions of the compounds with metal ions and Aβ may result in enhanced regulation of metal-induced Aβ aggregation, suggesting that metal-Aβ interactions could be a key parameter for metal-induced Aβ aggregation possibly occurring in the actual diseased brain, although an understanding of the mechanism is not necessary to practice the invention. Further, these studies show the ability of these classes of compounds to target metal-Aβ species and control their interaction/reactivity.

### Effects of 3b on Metal-Aβ Interaction Using Aβ Species in Human AD Brain Tissue Homogenates

The frontal cortex section of the human AD brain was selected because it generally contains high amounts of Aβ aggregates. Interestingly, upon 24 h treatment of human AD brain tissue homogenates with 3b, more gel-permeable and lower MW Aβ species were visualized by Western blotting with 6E10 (FIG. 5, bottom). These results show that 3b could fragment existing Aβ aggregates surrounded by metal ions in vivo (low μM Cu and Zn was found in brain tissue samples by ICP-MS, FIG. 5, bottom). Thus, the studies using human AD brain tissue homogenates show that the molecules can target metal-Aβ species and show reactivity in heterogeneous environments, which demonstrates their use for investigating and regulating metal-Aβ species and events in vivo. Also, using these compounds, similar to in vitro disaggregation studies with synthetic Aβ peptide above, perturbing metal-Aβ interaction is contemplated to find use for disassembling Aβ aggregates in brain tissue.

### Modulation of Metal-Aβ Neurotoxicity In Vitro and in Living Cells

In addition to Aβ aggregation events, metal-Aβ interaction has been suggested to be associated with neurotoxicity including ROS generation (particularly, for redox-active metal ions). Samples containing CuII, Aβ, and either the compounds in cell-free solutions showed 70 to 80% lower H₂O₂ concentration using a horseradish peroxidase/Amplex Red assay. This reveals that the compounds can attenuate H₂O₂ production by Cu-Aβ and thus be classified as ROS regulators.

In the case of compounds 5a-5d, their antioxidative activity was measured using the trolox equivalent antioxidant capacity (TEAC) assay. The result indicated that the compounds can be better antioxidants than trolox (vitamin E) (approximately, 3-fold better than trolox).

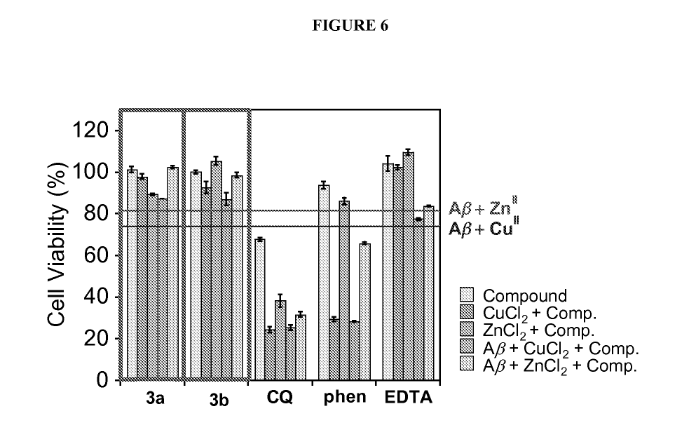

Preliminary cytotoxicity studies of the compounds in human SK-N-BE(2)-M17 (M17) neuroblastoma cells were carried out using a standard colorimetric assay for measuring cell growth (i.e., MTT assay; MTT=3-(4,5-dimethylthiazol-2-yl)-2,5-diphenyltetrazolium bromide). The results indicated no toxicity of 2a/b (up to 100 μM; they are less cytotoxic than CQ) and of 3a/b (up to 40 μM) with 1 equivalent of CuII or ZnII for 24 h. In addition, up to 200 μM, 5a-5d did not indicate any cytotoxicity in M17 cells. More importantly, compound 3a/b noticeably diminished toxicity induced by metal-Aβ (FIG. 6). Taken together, the ROS and cell studies support that the metal-Aβ interaction is part of the pathological processes in AD. Furthermore, the compounds, as controllers of metal-Aβ interaction, find use to reduce metal-Aβ toxicity and find use for in vivo investigations and therapies.

### In Vivo Biodistribution of Small Molecules

For investigating the biodistribution and confirmation of BBB crossing of the molecules, one structure 3b, was radiolabeled by tritiation of its precursor (FIG. 3). Healthy female C57B1/6 mice (n=18) were administered by bolus intravenous injection with [³H]-3b (2.5 μCi) each. The mice were divided into 3 groups of 6. Each group was sacrificed at 10 min, 60 min and 120 min post injection. Biodistribution was carried out by harvesting blood from cardiac puncture and tissue (liver, heart, kidneys, lungs, small intestine, muscle, spleen, feces, bladder, bone and brain). The brain was divided into four different sections (cerebellum, frontal cortex, parietal cortex and striatum). The tissue was digested and counted for accumulated activity.

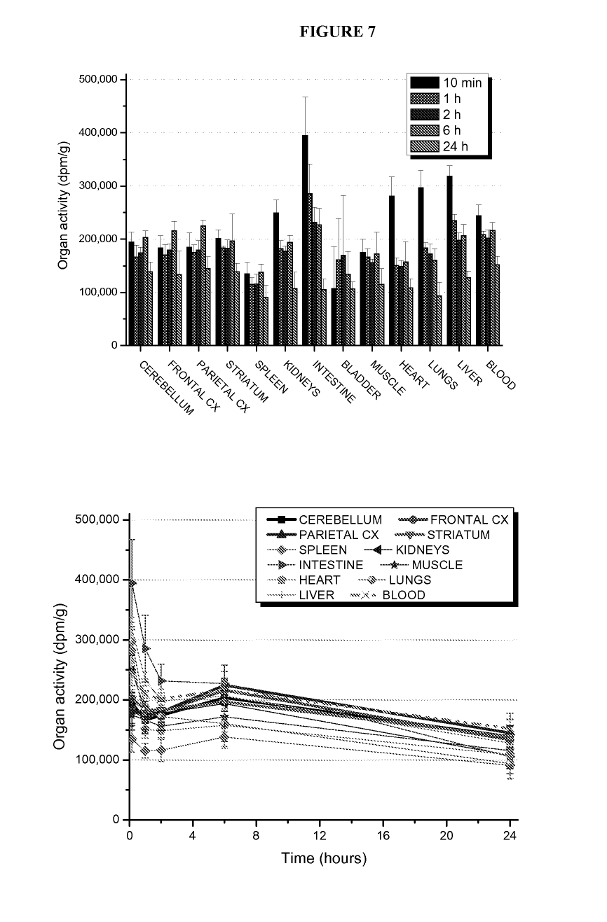

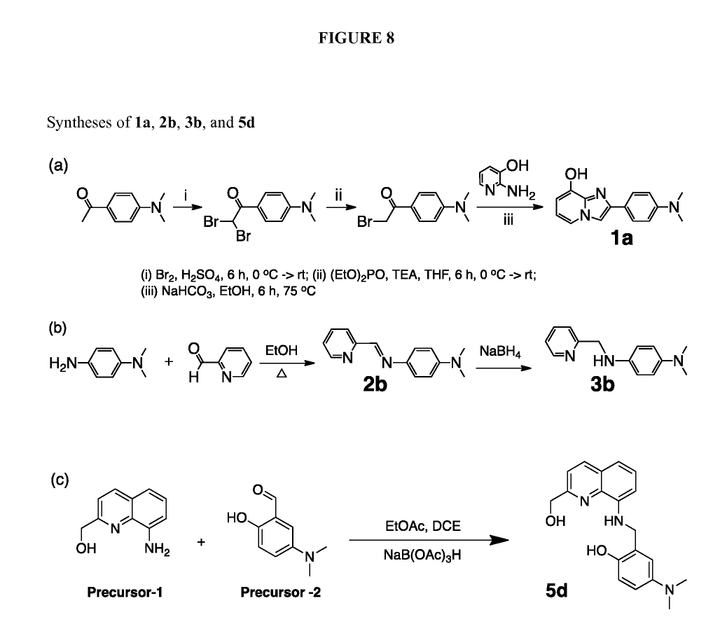

The plot of activity/g vs. organs (FIG. 7) shows the results. This compound clearly can cross the BBB. The brain uptake is relatively consistent within the time points chosen, for all four analyzed sections of the brain.

### Pharmaceutical Compositions and Dosing Considerations

Provided herein are compositions that comprise a therapeutically-effective amount of one or more of the compounds described above, formulated together with one or more pharmaceutically acceptable carriers (additives) and/or diluents, alone, or in combination with other therapeutic agents (i.e., configured for co-administration with other therapeutic agents). As described in detail below, the pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations. (See e.g., Martin, Remington's Pharmaceutical Sciences, 15th Ed., Mack Publ. Co., Easton, Pa. (1975)).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 0.1 per cent to about ninety-nine percent of active ingredient, preferably from about 5 per cent to about 70 per cent, most preferably from about 10 per cent to about 30 per cent.

In certain embodiments, a formulation comprises an excipient selected from the group consisting of cyclodextrins, celluloses, liposomes, micelle forming agents, e.g., bile acids, and polymeric carriers, e.g., polyesters and polyanhydrides; and a therapeutic compound. In certain embodiments, an aforementioned formulation renders orally bioavailable a compound.

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of this invention suitable for parenteral administration comprise one or more compounds in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the compounds are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian can determine and prescribe the effective amount of the pharmaceutical composition required. For example, the physician or veterinarian can start doses of the compounds employed in the pharmaceutical composition at levels lower than that required in order to achieve the desired therapeutic effect and gradually increase the dosage until the desired effect is achieved.

In general, a suitable daily dose of a compound will be that amount of the compound which is the lowest dose effective to produce a therapeutic effect. Such an effective dose will generally depend upon the factors described above. Generally, oral, intravenous, intracerebroventricular and subcutaneous doses of the compounds for a patient will range from about 0.0001 to about 200 mg per kilogram of body weight per day. In some embodiments, the compounds are administered at about 0.01 mg/kg to about 200 mg/kg, at about 0.1 mg/kg to about 100 mg/kg, or at about 0.5 mg/kg to about 50 mg/kg. When the compounds described herein are co-administered with another agent, the effective amount may be less than when the agent is used alone.

If desired, the effective daily dose of the active compound may be administered as two, three, four, five, six or more sub-doses administered separately at appropriate intervals throughout the day, optionally, in unit dosage forms.

While it is possible for a compound to be administered alone, it may be desired to administer the compound as a pharmaceutical formulation (composition).

### Medical and Research Uses

The compounds and compositions described herein find use in a variety of therapeutic, diagnostic (including screening), and research applications. The compounds, compositions, and methods find particular use in the study, detection, diagnosis, monitoring, treatment, or prevention of neurological diseases or conditions. In particular, any disease or condition associated with the presence of or amount of amyloid-β (Aβ) plaques as a pathogenic marker find use with the methods, compounds, and compositions described herein.

Among the neurological diseases and conditions are various forms of dementia. Dementia is not a single disease, but rather a non-specific illness syndrome (i.e., set of signs and symptoms) in which affected areas of cognition may be memory, attention, language, and problem solving. It is normally required to be present for an extended period of time (e.g., 6 months) to be diagnosed, however the detection of relevant biological markers, including those described herein, can provide for an earlier diagnosis. Some of the most common forms of dementia are: Alzheimer's disease, vascular dementia, frontotemporal dementia, semantic dementia and dementia with Lewy bodies. It is possible for a patient to exhibit two or more dementing processes at the same time, as none of the known types of dementia protects against the others.

There are many other medical and neurological conditions in which dementia only occurs late in the illness, or as a minor feature. For example, a proportion of patients with Parkinson's disease develop dementia. When dementia occurs in Parkinson's disease, the underlying cause may be dementia with Lewy bodies or Alzheimer's disease, or both. As such, such patients may be subjected to diagnostic or therapeutic methods described herein.

In some embodiments, the disease or condition is diabetes. Aggregation of human islet amyloid polypeptide (hIAPP) into cytotoxic β-sheet oligomers and amyloid plaques is considered a key event in the pancreatic β-cell degeneration in type II diabetes (see e.g., Sellin et al., Biophysical Chem. 150, 73-79 (2010); Edginton, Biotechnology, 12: 591-594 (1994), herein incorporated by reference in their entireties). A number of studies have reported that people with diabetes are more likely to develop dementia or Alzheimer's disease. As such, in some embodiments, the compounds described herein are administered to subjects with, or at risk for, type II diabetes for diagnostic, therapeutic, or research applications.

Depending on the disease or condition exhibited by the subject, the compounds and compositions described herein may be partnered with other diagnostic or therapeutic agents. For example, for treatment of Alzheimer's disease, the compounds may be co-administered with one or more of acetylcholinesterase inhibitors (e.g., Tacrine, Rivastigmine, Galantamine, and Donepezil) or NMDA receptor antagonists (e.g., memantine).

### EXAMPLES

### Materials and Procedures

All reagents were purchased from commercial suppliers and used as received unless stated otherwise. The compound 2-bromo-1-(4-(dimethylamino)phenyl)ethanone was synthesized by previously reported methods (Diwu, Z.; Beachdel, C.; Klaubert, D. H. Tetrahedron Lett. 1998, 39, 4987-4990.). NMR spectra were recorded on a Varian 400 spectrometer and IR spectra were obtained on a Perkin-Elmer Spectrum BX FT-IR instrument. Optical spectra were collected on Agilent 8453 UV-visible spectrophotometer.

2-[4-(dimethylamino)phenyl]imidazo[1,2-α]pyridine-8-ol (1a). 2-bromo-1-[4-(dimethylamino)phenyl]ethanone (300 mg, 1.2 mmol) and 2-amino-3-hydroxypyridine (114 mg, 1.0 mmol) were combined in EtOH (6 ml) under nitrogen. The reaction mixture was slowly heated to 75° C. (starting at 45° C. and temperature was increased by 10° C. every 20 minutes) and stirred under reflux for 2 h. Sodium bicarbonate (150 mg, 1.8 mmol) was added after mixture was cooled. The resulting mixture was slowly heated to 75° C. and stirred under reflux for 4 h. After mixture was cooled, the reaction was diluted 1:1 with 7 ml water. Product is pH sensitive and precipitated out upon addition of 5 N NaOH. The precipitate was collected by filtration, washed with water, and air dried under vacuum to afford a dark red/brown solid. The crude product was purified by gradient column chromatography (SiO₂, EtOAc:Hx=1:1→EtOAc:Hx:MeOH=1:1:0.5) and washed several times with CH₂Cl₂ yielding a light brown product (99 mg, 0.39 mmol, 39%). ¹H NMR (400 MHz, d₆-DMSO)/δ (ppm): 2.90 (6H), 6.41 (1H, d, J=7.6), 6.60 (1H, t, J=7.6), 6.74 (2H, d, J=9.2), 7.74 (2H, d, J=8.8), 7.92 (1H, d, J=6.0), 8.09 (1H, s). ¹³C NMR (100 MHz, d₆-DMSO)/δ (ppm): 150.3, 146.4, 144.5, 140.1, 128.9, 122.6, 118.3, 112.7, 108.4, 104.6, 40.5. HRMS: Calcd for [M+H]⁺, 254.1293; Found, 254.1293. UV-visible in EtOH: (λₘₐₓ, ε (M⁻¹cm⁻¹)) (282, 2.6×10⁴), (322, 2.1×10⁴). FTIR (KBr, cm⁻¹): 3433 (s, br), 3127 (vw), 3105 (vw), 2890 (vw), 2800 (vw), 1613 (vs), 1547 (m), 1507 (s), 1491 (s, sh), 1442 (m), 1429 (m, sh), 1378 (m), 1359 (s), 1328 (w), 1298 (m), 1275 (m), 1256 (w), 1227 (w), 1206 (m), 1173 (w), 1131 (vw), 1098 (w), 1077 (vw), 1006 (vw), 993 (vw), 977 (vw), 962 (vw), 946 (w), 915 (vw), 901 (vw), 885 (vw), 855 (vw), 823 (w), 773 (w), 747 (m), 733 (w), 718 (vw), 669 (vw), 654 (vw), 638 (vw), 608 (vw).

N¹,N¹-dimethyl-N⁴-(pyridin-2-ylmethylene)benzene-1,4-diamine (2b). Picolinaldehyde (210 μL, 2.2 mmol) was added into EtOH (dry, 3 mL, stirred at room temperature under N₂ for 5 min) of N¹,N¹-dimethylbenzene-1,4-diamine (300 mg, 2.2 mmol). The solution was refluxed for 30 min and was cooled to room temperature. The crude compound was obtained by addition of Et₂O (5 mL), collected, washed with Et₂O three times, and dried in vacuo, yielding a pure green product (450 mg, 2.0 mmol, 91%). ¹H NMR (400 MHz, CD₂Cl₂)/δ (ppm): 6.8 (2H, J=8.8), 7.35-7.29 (3H, m), 7.77 (1H, t, J=7.6), 8.17 (1H, d, J=8.0), 8.63 (8.64 (sh), 2H). ¹³C NMR (100 MHz, d₆-DMSO)/δ (ppm): 155.4, 155.3, 150.1, 149.5, 139.4, 136.5, 124.3, 122.9, 121.3, 112.6, 40.6. HRMS: Calcd for [M+Na]⁺, 248.1164; Found, 248.1170. UV-visible in EtOH: (λₘₐₓ, ε (M⁻¹cm⁻¹)) (398, 2.7×10⁴). FTIR (KBr, cm⁻¹): 3421 (w, br), 3130 (vw), 3049 (vw), 2915 (w), 2888 (w), 2841 (w), 1619 (s), 1591 (m), 1572 (vs), 1519 (s), 1465 (m), 1443 (w), 1428 (m), 1366 (s), 1346 (m, sh), 1301 (w), 1230 (m), 1212 (w, sh), 1167 (s), 1142 (w), 1122 (w), 1069 (w), 989 (w), 970 (w), 948 (w), 927 (vw), 902 (vw), 876 (vw), 811 (s), 777 (w), 746 (w), 741 (w), 720 (vw), 632 (vw), 629 (vw), 622 (vw), 614 (vw), 611 (vw).

N¹,N¹-Dimethyl-N⁴-(pyridin-2-ylmethyl)benzene-1,4-diamine (3b). Sodium borohydride (NaBH₄, 100 mg, 2.6 mmol) was added into a methanol solution (3 mL, cooled to 0° C.) of the imine precursor, N¹,N¹-dimethyl-N⁴-(pyridin-2-ylmethylene)benzene-1,4-diamine (2b (2), 100 mg, 0.39 mmol). The solution was stirred for 5 min at 0° C. After 30 min to room temperature, the reaction was quenched by water (10 mL) followed by extraction with Et₂O (10 mL) three times. The crude product was purified by column chromatography (SiO₂, ethyl acetate:hexanes=1:1) yielding a light product (for 3b: R_{f}=0.23, 65 mg, 0.29 mmol, 74%). ¹H NMR (400 MHz, CDCl₃)/δ (ppm): 2.80 (6H, s), 4.40 (4.23 (sh), 3H, s), 6.63 (2H, d, J=8.8 Hz), 6.72 (2H, d, J=8.8 Hz), 7.13-7.16 (1H, m), 7.33 (1H, d, J=8.0 Hz), 7.60 (1H, td, J=8.0 Hz, J=2.0 Hz), 8.56 (1H, d, J=4.4 Hz). ¹³C NMR (100 MHz, CDCl₃)/δ (ppm): 159.2, 149.2, 144.2, 140.4, 136.6, 122.0, 121.6, 115.8, 114.5, 50.4, 42.2. HRMS: Calcd for [M+Na]⁺, 250.1320; Found, 250.1312.

4-(Dimethylamino)-2-(((2-(hydroxymethyl)quinolin-8-yl)amino)methyl)phenol (5d). A dry ethyl acetate solution (8 mL) of the precursor-1 (Lim, M. H.; Wong, B. A.; Pitcock, Jr., W. H.; Mokshagundam, D.; Baik, M. H.; Lippard, S. J. J. Am. Chem. Soc., 2006, 128, 14363-14373; Roth, R.; Erlenmeyer, H. Helv. Chim, Acta., 1954, 37, 1064-1068.) (173.9 mg, 0.99 mmol) and the precursor-2 (Waibel, M.; Hasserodt, J. Tetrahedron Letters, 2009, 50, 2767-2769.) (163.5 mg, 0.99 mmol) was stirred overnight at room temperature. After drying, the residue was dissolved in dichloroethane (8 mL). To this solution was added NaB(OAc)₃H (419.7 mg, 1.98 mmol) and the reaction solution was stirred for 24 h at room temperature. After removing solvents, the salts of this solution were removed by fresh column (SiO₂, 1:5 Hexane/Ethyl acetate, R_{f}=0.47). The orange powder was obtained by recrystallization (1:1 HCl/H₂O, 198.2 mg, 0.50 mmol, 50.5%). ¹H NMR (400 MHz, CD₃OD)/δ (ppm): 8.73 (1H, d, J=8.4 Hz), 7.92 (1H, d, J=8.8 Hz), 7.80 (1H, d, J=8.0 Hz), 7.67 (1H, t, J=8.0 Hz), 7.61 (1H, d, J=2.8 Hz), 7.55 (2H, m), 7.03 (1H, d, J=8.8 Hz), 5.12 (2H, s), 4.74 (2H, s), 3.16 (6H, s). ¹³C NMR (100 MHz, d₆-DMSO)/δ (ppm): 159.17, 156.03, 141.78, 139.81, 134.53, 133.70, 127.92, 127.77, 126.17, 121.41, 120.73, 119.58, 115.82, 115.13, 106.21, 63.19, 45.99, 42.21. ESI(+)MS: Calcd for C₁₉H₂₁N₃O₂ ([M+H]⁺), 324.1; Found, 324.2. HRMS: Calcd for [M+H]⁺, 324.1707; Found, 324.1697. Anal. Calcd for C₁₉H₂₃N₃O₂Cl₂ (396.31): C, 57.58; H, 5.85; N, 10.60; Cl, 17.89. Found: C, 54.81; H, 5.85; N, 9.85; Cl, 18.91%.

Radiolabeling of Compounds

The following description provides exemplary labeling methods for labeling a compound with ¹²³I and ¹⁸F.

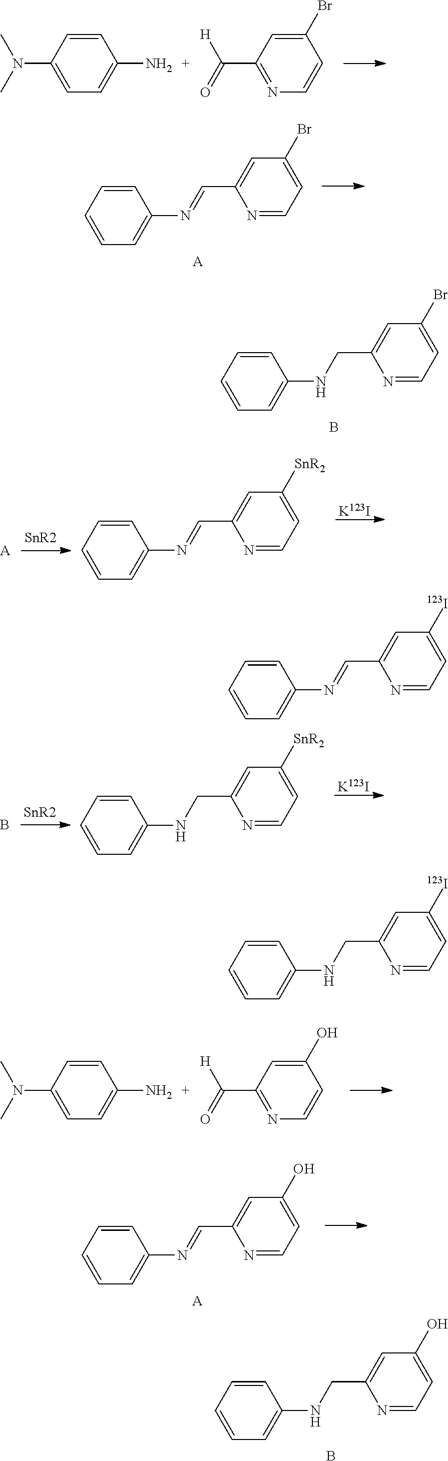

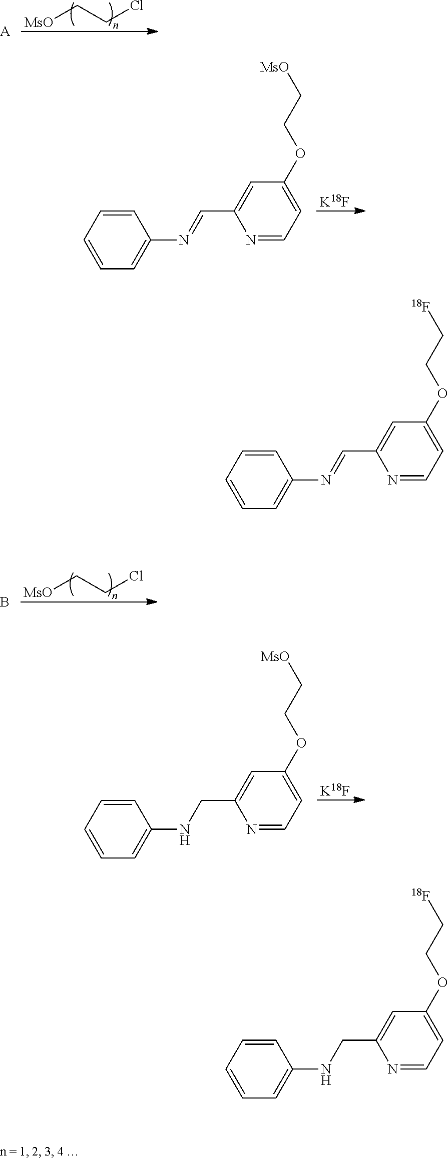

Based on the strategies above, a library of compounds can be synthesized by mix and match of precursors (and also substituted precursors) for the compounds described herein (substituted precursor refers to having additional groups instead of H in the pyridine carboxaldehyde ring or the aniline ring such as alkyl chains, etc.). The compounds can be labeled with multiple labels simultaneously as well.

In Vivo Activity of Small Molecules

During the development of embodiments of the technology provided herein, experiments were conducted to verify the efficacy of pharmaceutical compositions according to the technology by testing in a murine model. Pharmaceutical compositions comprising compounds described herein were administered every two days at 1 mg/kg (0.1 ml/10 g body weight) to four female Tg2576 hAPP-transgenic mice (18 months old) for two months (total 30 times in 60 days) by oral gavage.

Efficacy was evaluated after drug administration. Tissue slices 12 mm long were prepared from cerebrums of Tg2576 mice by a procedure comprising rapid freezing and cutting using a cryostat. To verify the degree of amyloid beta accumulation, the brain tissue slices were stained by APP/Aβ17-24-immunospecific antibody 4G8. In addition, to identify the amount of mature amyloid plaques that were generated in the presence of Apolipoprotein E (ApoE), sample were stained with Congo red.

Data collected show that pharmaceutical compositions according to the technology (e.g., compound 3b) and related methods of treatment reduced Aβ immunoreactivity and congophilicity (p<0.01) significantly relative to treatment with control vehicle alone. Accordingly, these molecules control amyloidopathogenesis (e.g., by control of amyloid plaque formation and/or promotion of their degradation) in a subject (e.g., a mammal such as a human, mouse, etc., e.g., as shown in the model Tg2576 hAPP-transgenic mouse).

We claim:

1-30. (canceled)

31. A compound comprising:
a) a metal chelation component; and
b) an amyloid β interaction component.

32. The compound of claim 31 wherein the compound has a structure according to: 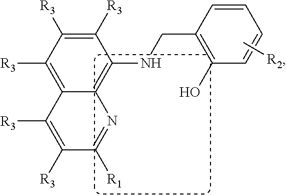
wherein:
i) R₁ is selected from the group consisting of H, OH, CH₃, CO₂CH₃, CO₂H, CH₂OH, F, O-glucose, ¹⁸F, I, ¹²³I, ¹²⁴I, ¹²⁵I, ⁷⁶Br, and ⁷⁷Br;
ii) R₂ is selected from the group consisting of H, OH, NH₂, NH(CH₃), N(CH₃)₂, F, O-glucose, ¹⁸F, I, ¹²³I, ¹²⁴I, ¹²⁵I, ⁷⁶Br, and ⁷⁷Br; and
iii) R₃ is each independently selected from the group consisting of H, OH, NH₂, NH(CH₃), N(CH₃)₂, (CH₂)ₙCH₃ (n=1-10), F, O-glucose, ¹⁸F, I, ¹²³I, ¹²⁴I, ¹²⁵I, ⁷⁶Br, and ⁷⁷Br.

33. The compound of claim 31, wherein the compound has a structure according to:

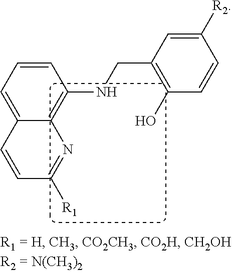

34. The compound of claim 31 wherein the compound has a structure according to: 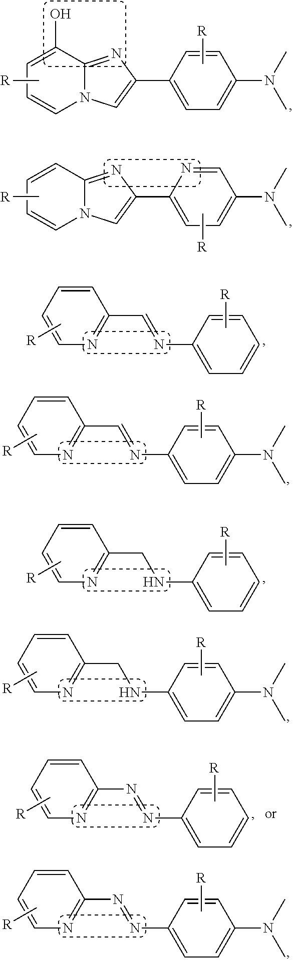
wherein each R is independently selected from the group consisting of H, I, F, CH₃, ¹⁸F, I, ¹²³I, ¹²⁴I, ¹²⁵I, ⁷⁶Br, and ⁷⁷Br.

35. The compound of claim 31 wherein the compound penetrates a blood-brain barrier of a human.

36. A method for treating a subject comprising administering the compound of claim 31 to a subject.

37. The method of claim 36 wherein said subject is a human.

38. The method of claim 36, wherein said subject has a neurological disease or condition.

39. The method of claim 38 wherein said neurological disease or condition has amyloid-β (Aβ) plaques as a pathological marker.

40. The method of claim 38 wherein said neurological disease or condition is Alzheimer's disease.

41. The method of claim 36 wherein said subject has type II diabetes.

42. The compound of claim 31 further comprising a label.

43. The compound of claim 42 wherein said label is a radionuclide or a substitution bearing a radionuclide.

44. The compound of claim 42 wherein said label is a contrast agent.

45. A method for detecting a compound in a tissue or subject, the method comprising:
exposing a tissue or subject a compound according to claim 42; and detecting the presence of or accumulation of said compound in said tissue or subject.

46. The method of claim 45, wherein said tissue comprises neural tissue or pancreatic tissue.
